(19) United States
(12) Plant Patent Application Publication
(10) Pub. No.: US 2017/0347516 P1
(43) Pub. Date: Nov. 30, 2017

(54) Agapanthus plant named 'ATIsea'

(50) ;

(71) Applicant: Anthony Tesselaar, Silvan (AU)

(72) Inventor: Anthony Tesselaar, Silvan (AU)

(21) Appl. No.: 14/999,562

(22) Filed: May 26, 2016

(51) Int. Cl. A01H 5/02

(52) U.S. Cl. CPC A01H 5/02
USPC PLT/398

(57) ABSTRACT

A new and distinct Agapanthus cultivar named ‘ATIsea’ is disclosed, characterized by abundant production of flowering umbels on short plants. Flowers are dark blue. Plants are uniquely tolerant of both drought and damp conditions. The new variety is a Agapanthus, typically produced as an outdoor ornamental plant.

Latin name of the genus and species: Agapanthus praecox subspecies orientalis.

Variety denomination: ‘ATIsea’.

### BACKGROUND OF THE INVENTION

The new cultivar is the product of chance discovery. The new variety originated as a naturally occurring mutation of the patented variety of Agapanthus ‘ATIblu’, U.S. Pat. No. 14,332.

The new variety was discovered by the inventor, Anthony Tesselaar, during 2010 in an outdoor planting of the parent variety in a commercial nursery in Silvan, Australia. After identifying the new variety as a potentially interesting selection, the inventor continued confidential observation of ‘ATIsea, finally selecting the variety for further propagation during 2011.

Asexual reproduction of the new cultivar ‘ATIsea’ was first performed by division in Silvan, Australia December 2011, on a very limited basis. Access to all plants was restricted, as plants were kept in a location not open to the public. Through subsequent propagation, multiple generations have been reproduced, which have shown that the unique features of this cultivar are stable and reproduced true to type.

### SUMMARY OF THE INVENTION

The cultivar ‘ATIsea’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘ATIsea.’ These characteristics in combination distinguish ‘ATIsea’ as a new and distinct Agapanthus cultivar:

1. Dark blue flowers.
2. Abundant inflorescence production, many flowering scapes per plant.
3. Short plant stature.
4. Tolerance for both drought and damp conditions.

### PARENT Comparison

Plants of the new cultivar ‘ATIsea’ are similar to the parent, Agapanthus ‘ATIblu’ in most horticultural characteristics. The new variety, however, has darker blue flowers than the parent variety.

### COMMERCIAL COMPARISON

Plants of the new cultivar ‘ATIsea’ are similar to the commercial variety Agapanthus ‘Storm Cloud’, unpatented. The new variety differs from ‘Storm Cloud in the following characteristics:

1. The new variety produces more flowering scapes then ‘Storm Cloud’.
2. The new variety tolerates wet conditions better than ‘Storm Cloud’.
3. Plants of the new variety are shorter and more compact than plants of ‘Storm Cloud’.

### BRIEF DESCRIPTION OF THE PHOTOGRAPHS

The accompanying photograph in FIG. 1 illustrates in full color a mass planting of ‘ATIsea’ grown in Silvan, Australia.

FIG. 2 illustrates in full color a close up of the flowers of ‘ATIsea’ and the parent variety ‘ATIblu’. Flowers of ATIsea’ are the darker umbel on the right.

FIG. 3 illustrates a close up of a flowering umbel of the new variety. The photographs were taken using conventional techniques and although colors may appear different from actual colors due to light reflectance it is as accurate as possible by conventional photographic techniques.

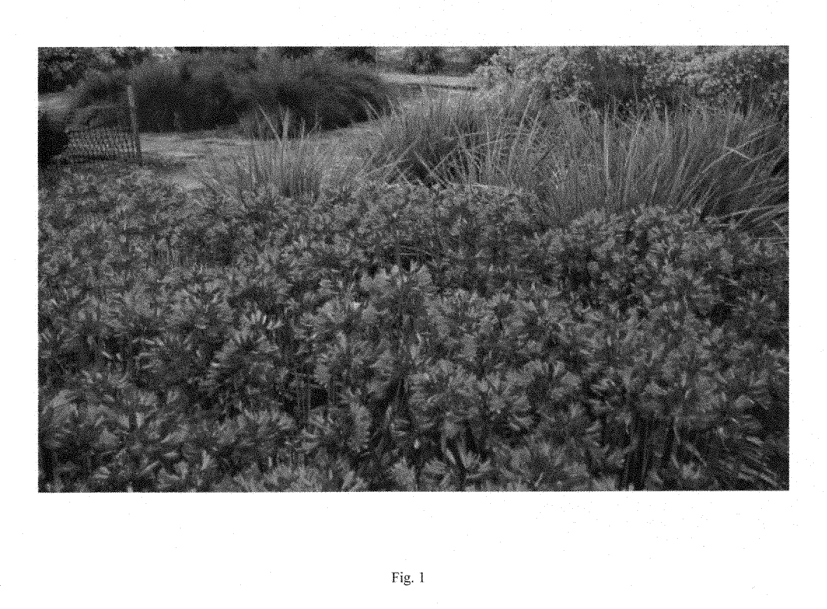

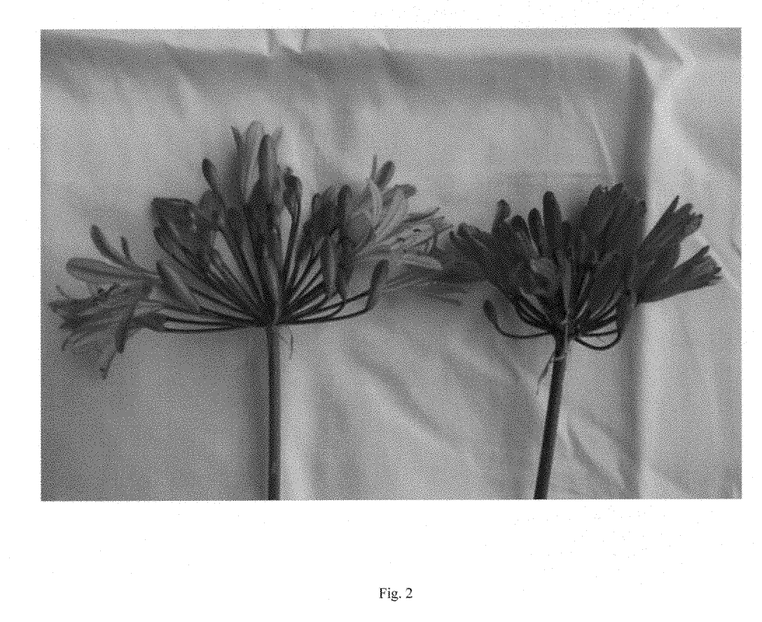

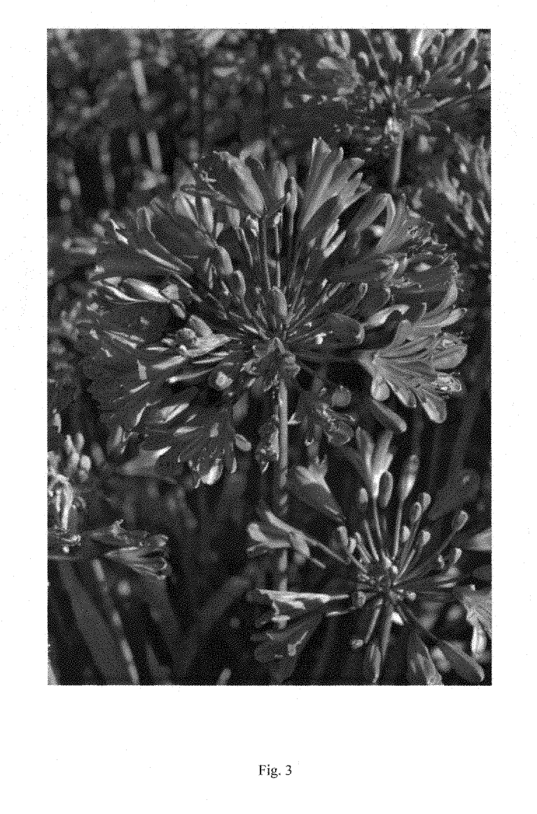

### DETAILED BOTANICAL DESCRIPTION

In the following description, color references are made to The Royal Horticultural Society Colour Chart 2007, except where general terms of ordinary dictionary significance are used. The following observations and measurements describe ‘ATIsea’ plants grown outdoors in Silvan, Australia. Plants are approximately 1 year old. Temperatures ranged from approximately 8° C. to 15° C. at night and 10° C. to 32° C. during the day. No artificial light, photoperiodic treatments were given to the plants. No chemical treatments were given to plants. Measurements and numerical values represent averages of typical plant types.
Botanical classification: Agapanthus praecox subsp. orientalis ‘ATIsea ’.
Propagation:
Root description: Thick, fleshy root system colored near Brown 199D and N199B.
Plant:
Growth habit: Clump forming tender perennial with arching, pendulous foliage.
Height: Flowering plane 700 to 850 mm.
Plant spread: Approximately 800 mm.
Growth rate: Rapid.
Stem: No stem, basal clump.
Growth pattern: The plant makes growth throughout most of the year but particularly in the Spring and Summer months. This growth is in the form of basal shoots or pups as well as some leaf extension.
Foliage:
Leaf:
   Average leaf length.—Approximately 230 mm.
   Average leaf width.—Approximately 12 mm.
   Color.—Coloration for both upper and lower surfaces. Immature: Near RHS Green 137B Mature: Near RHS Green 137B.
Flower:
Natural flowering season: Spring and Summer. Heavy flowering Spring and until mid-Summer. Sporadic flowering from mid-Summer until Fall.
Length of flowering period: Approximately 120 days.
Inflorescence type: Terminal Umbel with approximately 35 to 40 individual flowers.
Individual flower type: Single.
Flower profile: Funnelform.
Lasting quality: Inflorescence shows no significant signs of aging for approximately 8 weeks.
Bud:
   Shape.−Ovate.
   Color.—Near RHS Violet-Blue N89C.
Flower head size:
   Diameter.—150 mm.
   Stem length.—Approximate average 700 mm.
Individual flowers:
   Length.—Average 3.0 cm.
   Width.—Average 2.5 cm.
Tepals:
   Arrangement.—Rotate, overlapping.
   Length.—Average 28 to 30 mm.
   Width.—Average 8 to 10 mm.
   Quantity.—6 .
   Tepal lobe.—Length: Average 18 mm Width: Average 8 to 10 mm Shape: Linear Margin: Entire Base: Fused Apex: Mucronate, or rounded.
   Perianth tube.—Length: Approximately 8 mm Color: Inner surface: Near Violet-Blue 94D Outer surface: Near Violet-Blue 96C.
Flower color:
   When opening.—Inner surface: Near RHS Violet-Blue 94C Outer surface: Near RHS Violet-Blue 96B, apex slightly flushed Violet-Blue 90C.
   Fully opened.—Inner surface: Near RHS Violet-Blue 94D Outer surface: Near RHS Violet-Blue 96C.
   Fading.—Inner surface: Near RHS Violet-Blue 97C Outer surface: Near RHS Violet-Blue 96C.
Flower filament color: Near Violet-Blue 94D.
Flower anther color: Near Purple 79A.
Anther quantity: 6, each approximately 25 mm long.
Stamen protrusion: Protrudes very slightly past the floral lobe apex.
Pistil:
   Number.—1.
   Length.—Approximately 2.7 cm.
   Style color.—Near RHS Violet 88C.
   Stigma.—Shape: Linear. Color: Near RHS Violet 88A.
Pedicel:
   Length.—30 mm.
   Texture.—Glabrous.
   Color.—Near Green 137B, sometimes lightly flushed Violet 86B.
Fragrance: None.
Other characteristics:
Seed production: Seed vessels: approximately 10 mm long, 3 mm diameter. Colored near Yellow-Green N144D. 3 flat sides, 3 separate compartments. Moderate seed production. Seeds oblong, flat, approximately 2 mm long, colored near Black 202A.
Disease resistance: Neither resistance nor susceptibility to normal diseases and pests of Agapanthus have been observed.
Temperature tolerance: USDA zones 8 through 11.
Drought tolerance: Will tolerate considerable dryness once established. Plants are also tolerant of damp conditions.

What is claimed is:

1. A new and distinct cultivar of Agapanthus plant named ‘ATIsea’ as herein illustrated and described.
